(19) United States
(12) Patent Application Publication
(10) Pub. No.: US 2019/0328210 A1
(43) Pub. Date: Oct. 31, 2019

(54) COLONOSCOPE HANDLE ATTACHMENT DEVICE

(71) Applicant: Alpine Medical Devices, LLC, Carmel, CA (US)

(72) Inventor: Jan P. Kamler, Carmel, CA (US)

(21) Appl. No.: 16/394,412

(22) Filed: Apr. 25, 2019

Related U.S. Application Data
(60) Provisional application No. 62/663,449, filed on Apr. 27, 2018.

(51) Int. Cl. A61B 1/00; A61B 1/31

(52) U.S. Cl. CPC A61B 1/0014; A61B 1/31

(57) ABSTRACT

A colonoscope handle attachment device includes a body having arms sized to couple with the colonoscope and a hook that is sized to receive another portion of the colonoscope therein, such as the endoscope shaft. The hook defines a U-shaped channel that can be angled relative to a primary longitudinal axis of the body. However, the U-shaped channel can also be oriented as offset parallel to the primary longitudinal axis of the body. The configuration of the device allows an operator of the colonoscope, such as a physician, to have a free hand to perform other tasks inasmuch as the hook holds or couples the other portion or sub-component of the colonoscope near the handle of the colonoscope. At least portions of the body and the hook are at least semi-flexible to allow the device to releasably connect with the handle of the colonoscope.

### CROSS-REFERENCE TO RELATED APPLICATION

This application claims priority from U.S. Provisional Application Ser. No. 62/663,449, filed Apr. 27, 2018, the disclosure of which is incorporated herein by reference.

### TECHNICAL FIELD

The present disclosure relates generally to a device that is used during a colonoscopy or other procedure. More particularly, the present disclosure relates to a device that connects with a colonoscope. Specifically, the present disclosure relates to a colonoscope handle attachment device that enables the physician to more easily operate the colonoscope by enabling portions of the colonoscope or other components to connect with the handle attachment device.

### BACKGROUND

A colonoscopy is an exam used to detect changes or abnormalities in the large intestine (colon) and rectum. During a colonoscopy, a long, flexible tube (colonoscope) is inserted into the rectum. A tiny video camera at the tip of the tube allows the doctor to view the inside of the entire colon. If necessary, polyps or other types of abnormal tissue can be removed through the scope during a colonoscopy. Tissue samples (biopsies) can be taken during a colonoscopy as well.

Conventional colonoscopy techniques are made with reference to FIG. 1, FIG. 2, and FIG. 3.

As depicted in FIG. 1, during colonoscopy, a colonoscope is inserted through the anus 10 into the rectum 12 and then is advanced through the sigmoid colon 14, the descending colon 16, transverse colon 18 and the ascending colon 20 to the cecum 22. Then the colonoscope is withdrawn slowly and colonic mucosa is inspected. If necessary, additional procedures such as polypectomy (polyp removal), biopsy (sampling of mucosa, mass, or polyps), or submucosal injection, etc. are performed.

The goal of the gastroenterologist (endoscopist) is to examine the entire colon, and this requires intubation and inspection of the cecum 22. Intubation of the cecum 22 may take from several minutes up to an hour or more depending on the gastroenterologist's skills and experience and on the anatomy of the colon. About 1-% to 20% of colonoscopies are difficult, and U.S. data shows that between 3% and 12% of colonoscopies are incomplete in that the cecum is not reached. The main reasons for difficult or incomplete colonoscopies are angulations or loops of the tortuous colon, spasming of the colon or hypertrophy of the colonic wall due to diverticulosis, and acute angulations of the colon caused by adhesions after female pelvic surgeries or infections. The sigmoid colon is the most difficult part of the colon to negotiate, but any other area of the colon can be difficult to navigate as well depending on the etiology.

To advance the colonoscope to the cecum, the endoscopist insufflates the colon with air, pushes the colonoscope forward, pulls it back, deflects the tip of the scope with the help of knobs (up/down, left/right), torques the insertion part of the colonoscope right (clock-wise) and left (counter clock-wise), wiggles and shakes the colonoscope, and hooks the tip of it behind a mucosa fold and then withdraws it back to straighten the tortuous colonic segment. Water flushing is used not only to clean the colon from remaining debris, but also to fill the sigmoid colon and to straighten its loops. Endoscopists also use endoscopic assistants who push their hands on the abdomen from outside the body to reduce or prevent looping of the colonoscope.

As depicted in FIG. 2 and FIG. 3, to operate the colonoscope, the gastroenterologist typically holds the grip section or handle 210 of the colonoscope 250 with one hand (e.g., the left hand) and operates the two main knobs of the handle of the colonoscope (right/left angulation control know 333 and up/down angulation control know 331). The other hand (e.g., the right hand) can push, pull, and torque the shaft 211 of the colonoscope 250. When negotiating a difficult angulation of the colon, more precise control of the tip 227 of the colonoscope 250 can be needed. The tip 227 of the colonoscope could be secured in a certain position with the help of the right/left angulation lock 339 and up/down angulation lock 337 (activated by the other hand, e.g., the right hand).

The colonoscopy procedure can start with the colonoscope 250 being fully flexible, with the flexibility ring 235 loose. When the transverse colon or ascending colon is reached and 3-4 feet of the colonoscope are in, the colonoscope 250 may start looping. As a result, the hand holding the insertion section of the scope (e.g., the right hand) can be used to turn the flexibility adjustment ring 235 clockwise to stiffen the shaft of the colonoscope 250 to make it easier to traverse therethrough (e.g., to the cecum).

Operation of the colonoscope as described, however, can have several disadvantages. To begin, use of one hand to control both knobs 333, 331 is difficult because they have a spring therein that biases them to neutral. If the handle 210 of the scope 250 is held by the hand (e.g., the left hand), and the thumb is used to control one knob (333 or 331), the second knob (331 or 333) can often not be simultaneously held down or activated. While the thumb of the one hand (e.g., the left hand) can in some cases be used to fix both left/right and up/down angulation control knobs 331, 333, this may be difficult for an endoscopist with a medium-sized hand and virtually impossible for an endoscopist with a small hand. In this case, the other hand (e.g., the right hand) helps with the knobs 331, 333, but this leads to a loss of control over the shaft 211 of the scope 250. The endoscopist may then have to ask for help from the nurse or assistant to push or hold the colonoscope shaft 211 or try to switch quickly between the knobs 331, 333 and the shaft 211 of the scope with the right hand. This may lead to loss of the desired position of the tip 227 of the scope and/or slippage of the tip of the scope back. Repeat attempts at intubation of a difficult area of the colon lead to prolongation of the procedure, patient discomfort and operator frustration. An endoscopist may likewise face a similar situation when performing biopsy, polypectomy, etc., in an area of colonic angulation when the colon or its motility is pushing the colonoscope backwards. Again, both hands can be needed to hold the colonoscope in place and to secure the knobs and at the same time an additional hand is needed for biopsy forceps, a snare, an injector, etc.

### SUMMARY

As identified in FIG. 1 through FIG. 3, issues continue to exist with colonoscope devices and the difficulty associated with the doctor needing to manually manipulate multiple devices during the colonoscopy procedure. What is needed, therefore, is an improved method or device for operating a colonoscope. The present disclosure addresses this issue and other issues by providing a device that couples to the colonoscope, or likewise an endoscope, to provide more control to the doctor during the procedure.

In accordance with one aspect, an exemplary embodiment of the present disclosure may provide an attachment device for connection with a colonoscope comprising: a body extending along a longitudinal axis having a first arm and a second arm sized to secure the body to a colonoscope; and a hook coupled to a body adapted to carry a sub-component of the colonoscope or another component used during a medical procedure. This exemplary embodiment or another exemplary embodiment may further provide an inner surface of the hook defining a U-shaped channel. This exemplary embodiment or another exemplary embodiment may further provide a lower concave portion of the inner surface of the hook; and a hook axis, wherein the lower concave portion extends around the hook axis. This exemplary embodiment or another exemplary embodiment may further provide wherein the hook axis is oriented relative to the longitudinal axis at an angle in a range from about 45 degrees to about 75 degrees. This exemplary embodiment or another exemplary embodiment may further provide that the angle at which the hook axis is oriented relative to the longitudinal axis is about 60 degrees. This exemplary embodiment or another exemplary embodiment may further provide at least one detent extending from the inner surface of the hook. This exemplary embodiment or another exemplary embodiment may further provide a plurality of detents aligned in a row along a length of the inner surface of the hook adapted to bias the sub-component of the colonoscope or the another component towards a lower concave portion of the inner surface of the hook. This exemplary embodiment or another exemplary embodiment may further provide at least two rows of detents aligned along the length of the inner surface of the hook adapted to bias the sub-component of the colonoscope or the another component towards the lower concave portion of the inner surface of the hook. This exemplary embodiment or another exemplary embodiment may further provide that at least one detent in one of the at least two rows is elongated relative to the longitudinal axis. This exemplary embodiment or another exemplary embodiment may further provide that the first arm and the second arm defined is semi-annular configuration of the body; and a third arm and a fourth arm extend longitudinally along an exterior length of the body; and a transverse aperture formed in each of the third arm and the fourth arm adapted to receive a pin therethrough adapted to couple the attachment device to the colonoscope. This exemplary embodiment or another exemplary embodiment may further provide wherein the first arm includes: a top wall that extends forwardly to a corner; and a surface extending longitudinally from the corner that is concavely curved near a lower end thereof to position a terminal end wall forwardly from the surface. This exemplary embodiment or another exemplary embodiment may further provide wherein the second arm includes: an S-shaped wall that is longitudinally elongated from an upper end to a lower end that terminates at a terminal wall of the second arm, and the lower end is disposed forward from the upper end. This exemplary embodiment or another exemplary embodiment may further provide an integral connection of the hook to the body that offsets the hook off to a first side the attachment device in a cantilevered manner. This exemplary embodiment or another exemplary embodiment may further provide a forward end of the hook positioned rearward from a forward end of the body. This exemplary embodiment or another exemplary embodiment may further provide an upper end of the hook positioned below an upper end of the body. This exemplary embodiment or another exemplary embodiment may further provide a lower end of the hook positioned above a lower end of the body.

In accordance with another aspect, an exemplary embodiment of the present disclosure may provide a method comprising: coupling a body of an attachment device to a first portion of a colonoscope; and coupling a second portion of the colonoscope to a hook on the attachment device. This exemplary embodiment or another exemplary embodiment may further provide moving the second portion of the colonoscope towards a concave inner surface on the hook; and precluding movement of the second portion of the colonoscope relative to the concave inner surface of the hook. This exemplary embodiment or another exemplary embodiment may further provide orienting an axis of the second portion of the colonoscope coupled to the hook at an angle in a range from about 45 degrees to 75 degrees relative to a longitudinal axis of the body. This exemplary embodiment or another exemplary embodiment may further provide connecting a secondary component to an exterior surface of one of the body and the hook.

### BRIEF DESCRIPTION OF THE SEVERAL VIEWS OF THE DRAWINGS

A sample embodiment of the disclosure is set forth in the following description, is shown in the drawings, and is particularly and distinctly pointed out and set forth in the appended claims. The accompanying drawings, which are fully incorporated herein and constitute a part of the specification, illustrate various examples, methods, and other example embodiments of various aspects of the disclosure. It will be appreciated that the illustrated element boundaries (e.g., boxes, groups of boxes, or other shapes) in the figures represent one example of the boundaries. One of ordinary skill in the art will appreciate that in some examples one element may be designed as multiple elements or that multiple elements may be designed as one element. In some examples, an element shown as an internal component of another element may be implemented as an external component and vice versa. Furthermore, elements may not be drawn to scale.

FIG. 1 shows the anatomy of the large intestine (colon).

FIG. 2 is a perspective view of a lower portion of a colonoscope with a flexibility adjustment ring.

FIG. 3 is a perspective view of a colonoscope.

FIG. 4A is a first side perspective view of an exemplary colonoscope handle attachment device on a colonoscope.

FIG. 4B is a second side perspective view of the exemplary colonoscope handle attachment device of FIG. 4A attached to the colonoscope.

FIG. 5A is a top plan view of the colonoscope handle attachment device.

FIG. 5B is a bottom plan view of the colonoscope handle attachment device.

FIG. 5C is a first side perspective view of the colonoscope handle attachment device.

FIG. 5D is a second side perspective view of the colonoscope handle attachment device.

FIG. 6 is a top plan view of the colonoscope handle attachment device having a pin connected thereto for securing the device to the colonoscope.

FIG. 7 is a perspective view on another embodiment of an exemplary colonoscope handle attachment device.

FIG. 8A is a top perspective view of another exemplary colonoscope handle attachment device.

FIG. 8B is a first side elevation view of the exemplary colonoscope handle attachment device of FIG. 8A.

FIG. 8C is a bottom perspective view of the exemplary colonoscope handle attachment device of FIG. 8A.

FIG. 8D is a second side elevation view of the exemplary colonoscope handle attachment device of FIG. 8A.

FIG. 8E is a top plan view of the exemplary colonoscope handle attachment device of FIG. 8A.

FIG. 8F is a front elevation view of the exemplary colonoscope handle attachment device of FIG. 8A.

FIG. 8G is a bottom plan view of the exemplary colonoscope handle attachment device of FIG. 8A.

FIG. 8H is a rear elevation view of the exemplary colonoscope handle attachment device of FIG. 8A.

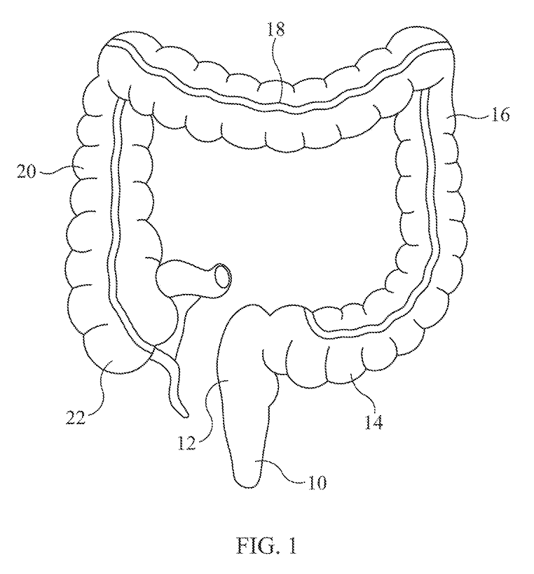

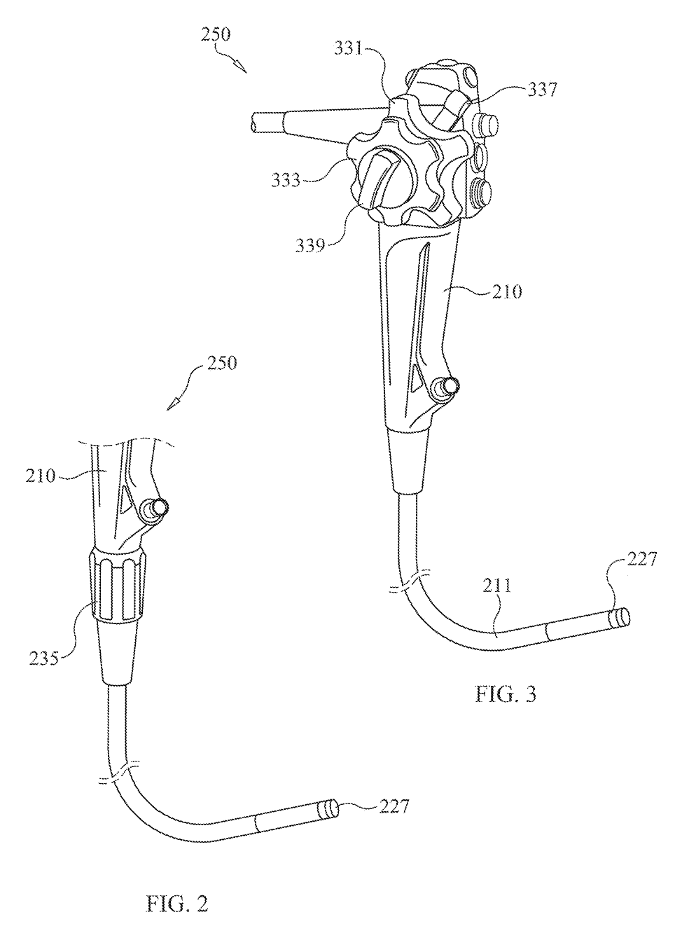

### DETAILED DESCRIPTION

A colonoscope handle attachment device is shown throughout the figures. The various embodiments of the colonoscope handle attachment device are configured to connect with an colonoscope and enable portions of the colonoscope to be carried by the attachment device that would conventionally be carried, supported, or held by the physician performing a medical procedure, such as a colonoscopy or endoscopy. The embodiments of the attachment devices may assist to reduce fatigue physician by alleviating the task of holding various portions of the colonoscope with two hands. The device's capability to reduce physician fatigue should enable the physician using the attachment device of the present disclosure to perform more procedures in a given day, which in turn may lead to increase profits for medical organization employing the physician or the medical facility within which the procedure is performed. The attachment device may also “free up” (i.e., make available) one hand of the physician so they can perform additional tasks with the hand that is now free but would have otherwise been required to hold a portion of the colonoscope absent the attachment device of the present disclosure.

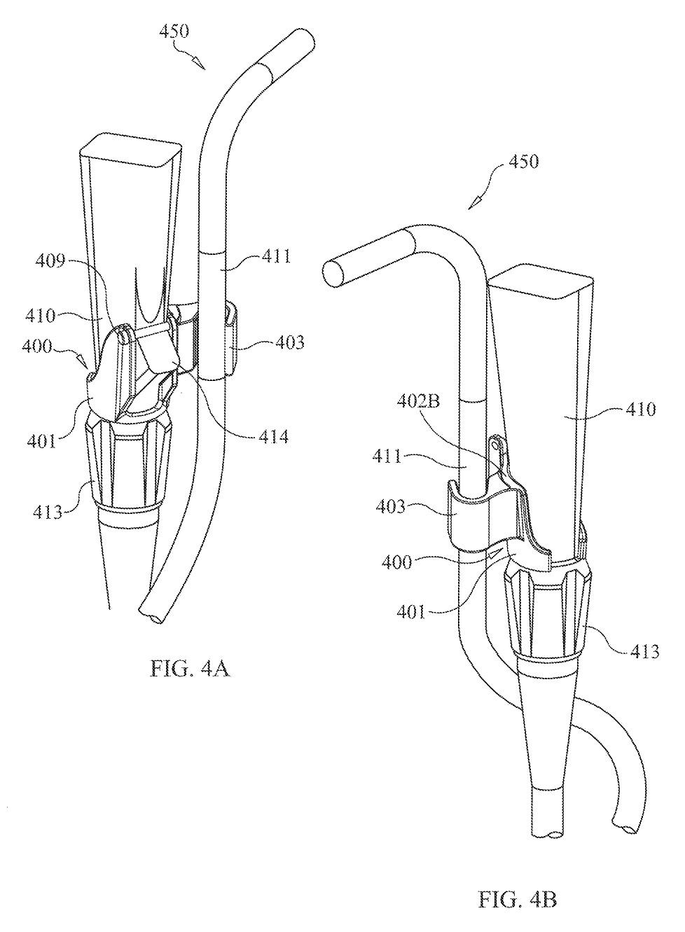

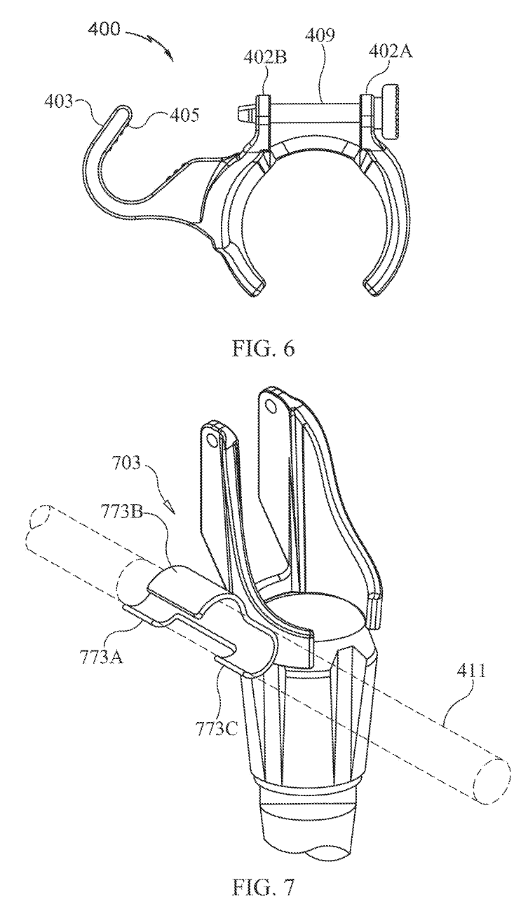

Referring to FIG. 4A and FIG. 4B, an exemplary attachment device 400 for a colonoscope 450 includes a c-shaped ring 401 for attachment to an endoscope handle 410 (e.g., directly above the scope flexibility adjustment ring 413). Parallel arms 402A, 402B can extend from the c-shaped ring 401 towards the proximal end of the handle 410. A pin 409 can be positioned between the arms 402A, 402B (e.g., so as to attach to the colonoscopy biopsy channel port 414 of the endoscope; see FIG. 6). The attachment device 400 can further include a hook 403 (e.g., extending from one of the arms 402B) for attachment around the shaft 411 of the endoscope (e.g. around the insertion section of the endoscope).

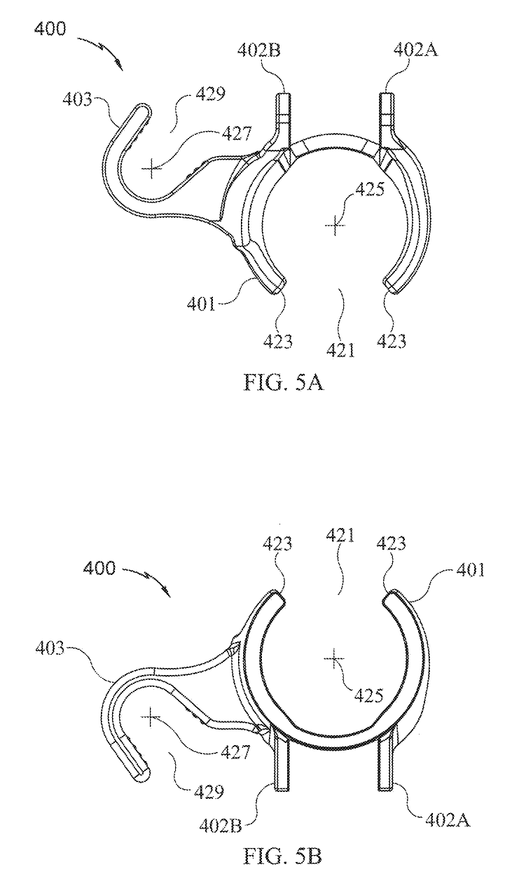

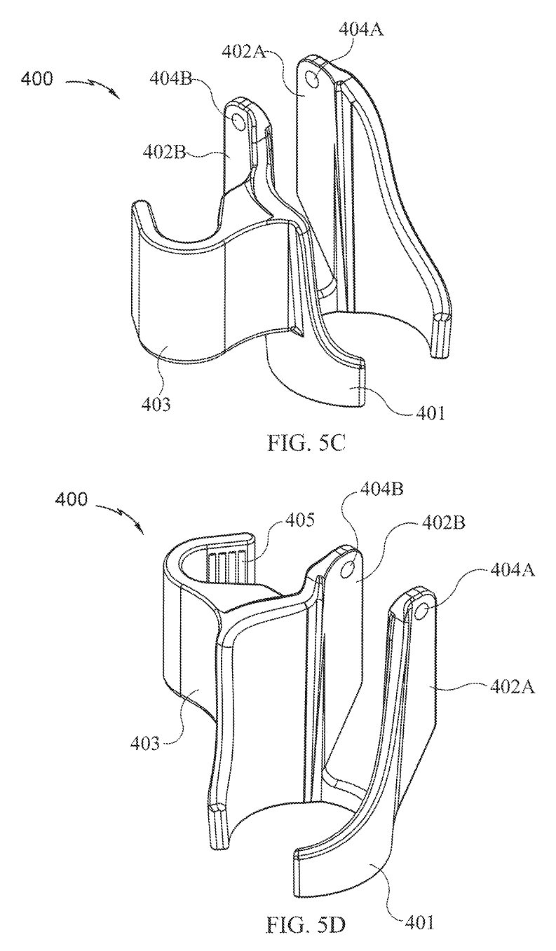

FIG. 5A and FIG. 5B depicts the attachment device 400 without an attachment pin 404 and FIG. 6 is shown with the pin 409. The device 400 thus includes three main parts: the c-shaped ring 401, the parallel arms 402A, 402B, and the hook 403.

The c-shaped ring 401 can be semicircular and extend about 270 degrees to about 320 degrees around defining an opening 421 defined between terminal ends 423. The ring 401 can be configured to anchor the device 400 around the distal part of the colonoscope handle just below the instrument channel port (e.g., the biopsy channel). In the case of a variable stiffness colonoscope, the c-shaped ring 401 can attach between the biopsy channel and the flexibility adjustment ring. The c-shaped ring 401 can advantageously prevent the device 400 from moving proximally relative to the colonoscope.

Ring 401 includes a convex outer surface and a concave inner surface. The concave inner surface is centered about a center vertical axis 425. The inner diameter of the ring 401 measured through vertical axis 425 is complementary to the outer surface of the colonoscope.

The parallel arms 402A, 402B can extend in a cantilevered manner from the c-shaped ring 401 outwardly from the outer convex surface and proximally along both sides of the instrument channel. The arms 402A, 402B therein for passage of the pin 409, which can rest on the instrument channel port to prevent movement of the device 400 distally.

The hook 403 can be attached to the outer surface of the c-shaped ring 401 and one of the parallel arms 402B. The hook 403 can serve as an anchor or attachment mechanism to the shaft of the scope. The hook 403 can be, for example, substantially U-shaped with a central axis (along the opening of the hook 403) that extends parallel to the central axis of the c-shaped ring 401. In one embodiment, a length of the hook 403 can be approximately half the length of the device 400. Further, the hook 403 can include features thereon configured to enhance the grip of the hook 403 with the colonoscope. For example, the hook can include internal ridges 405 thereon to provide anchoring to the scope.

Hook 403 includes a convexly curved outer surface and a concavely curved inner surface centered around a vertical hook axis 427. Hook axis 427 is offset parallel to the vertical axis 425 of the c-shaped channel ring 401. Hook 403 defines an opening 429 that is oriented in a different direction than the opening 421 of the ring 401. Opening 429 generally faces the direction arms 402A, 402B.

The device 400 advantageously has a simple design that allows a quick attachment to the handle 410 of the colonoscope via the c-shaped ring 401. The hook 403 of the device 400 provides quick in and out attachment of the shaft 411 of the colonoscope.

In an exemplary method of use of the device 400 with a colonoscope, when the shaft 411 of the colonoscope is attached, a user's first hand (e.g., the left hand) can hold the handle 410 of the colonoscope and control one of the operating knob while the other hand (e.g., the right hand) can be free to operate the other control knob (because the shaft 411 of the scope 450 is supported by the hook 403 and therefore need not be held by the other hand). This allows for insertion and very fine deflection of the tip of the colonoscope (e.g., deflection in the up/down and left/right directions simultaneously. At the same time, movement of both hands (which can be on the handle 410) proximally towards the patient can advance the device 400 (and thus the shaft 411 of the scope 450) to provide advancement of the tip of the scope 450 forward through the angulated area of the colon. Simultaneous operation of both knows with both hands and forward movement of the scope 450 advantageously greatly facilitates negotiations of the toughest colonic angulations. Similarly, the device 400 can be used during withdrawal of the colonoscope 450. Fine control of the tip of the colonoscope with both hands operating the angulation control knobs can prevent interruption of the colon inspection.

In another exemplary method of use, the device 400 can be used to hold the colonoscope 450 in place with one hand (e.g., the left hand) while using the other hand (e.g., the right hand) to perform a medical procedure, such as a biopsy, polypectomy, or injections.

The device 400 can be single use and disposable. Use of the device 400 can markedly shorten the time of cecal intubation of any more or less skilled endoscopist can increase the rates of successful cecal intubation and thus increased detection of polyps.

The hook used for attachment to the colonoscope can have various shapes and/or features to aid in attachment. For example, FIG. 7 depicts another exemplary hook 703 that can be similar to hook 403 except that hook 703 includes three prongs 773A, 773B, 773C wherein two prongs 773A and 773C on one side with a cut-out therebetween and one central prong 773B on the other side. Additionally, while the hook 703 is shown rotated 90 degrees relative to the hook 403, it can be configured so as to have a central axis that is substantially parallel to the central axis (similar to hook 403) to aid in directly inserting the colonoscope proximally as the hand is moved proximally.

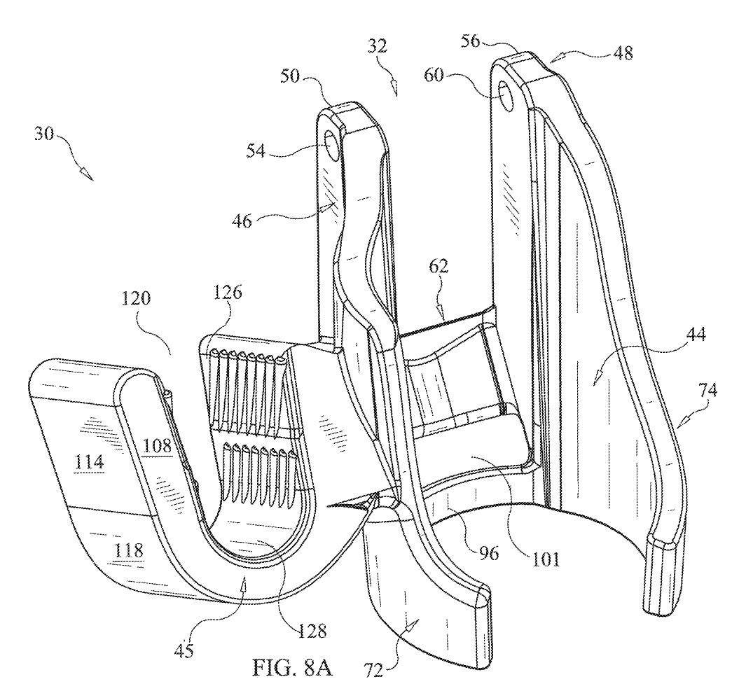

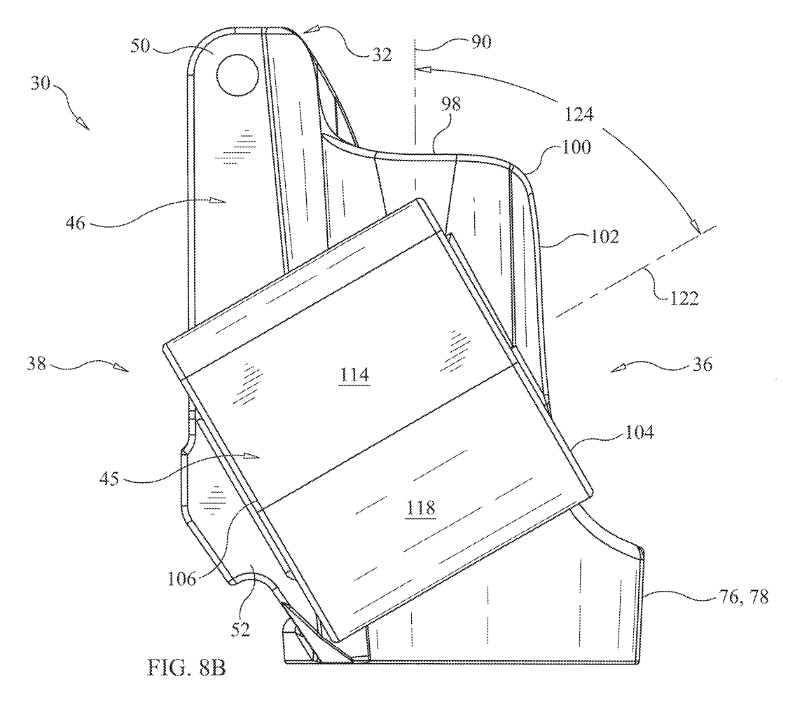

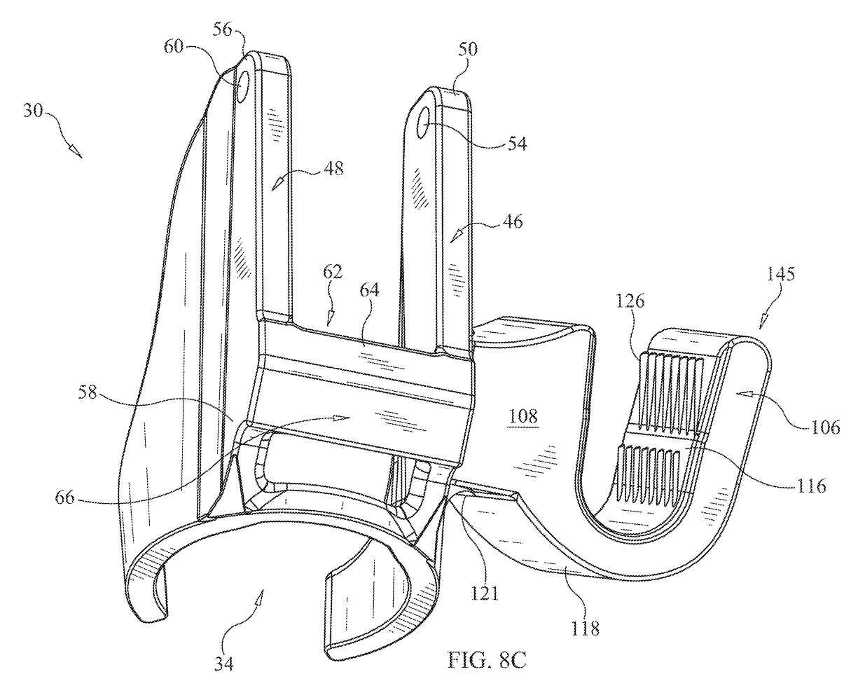

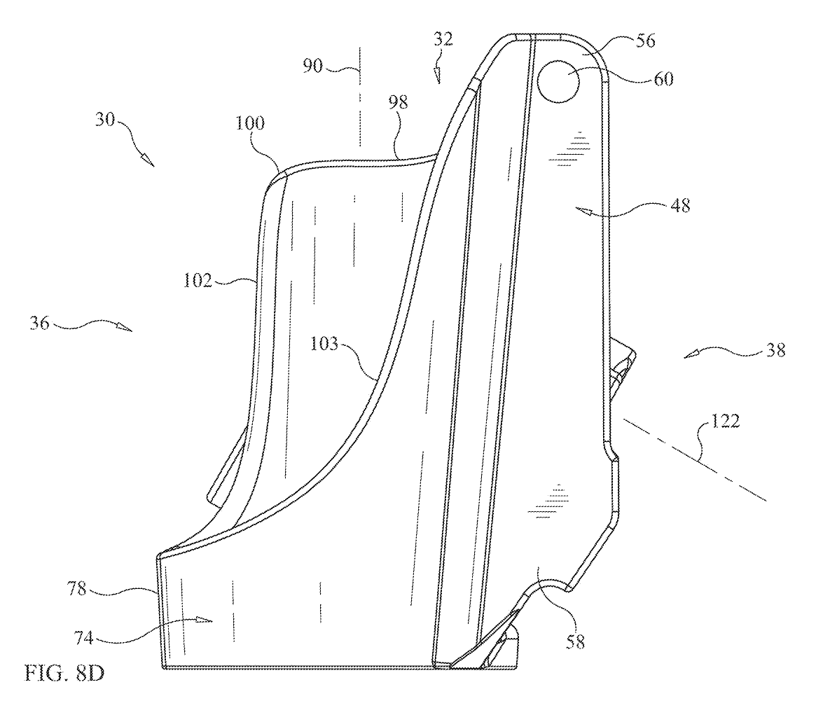

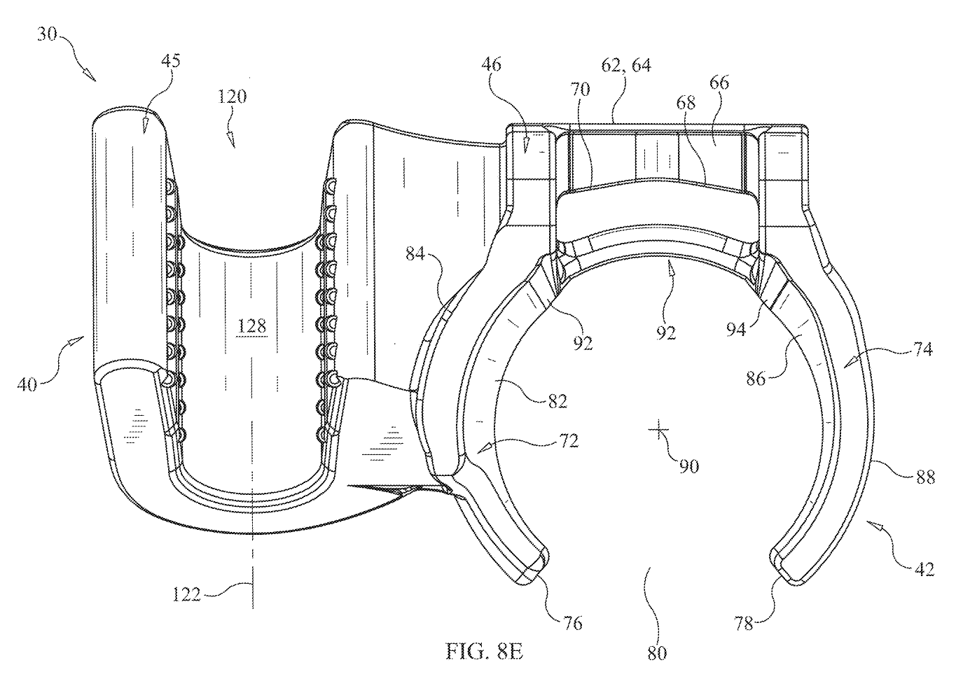

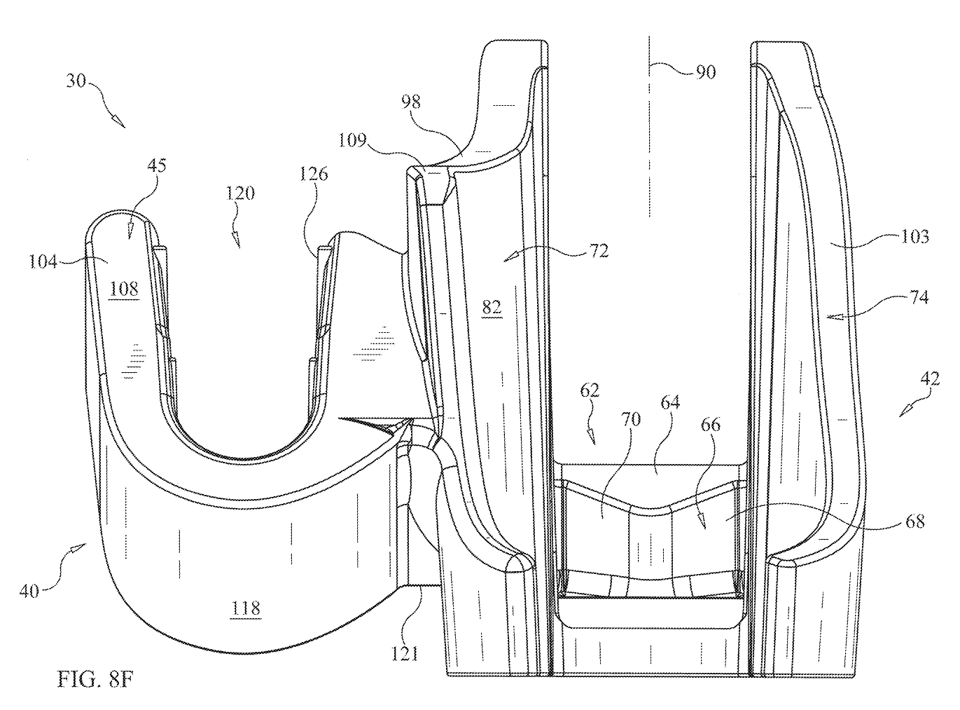

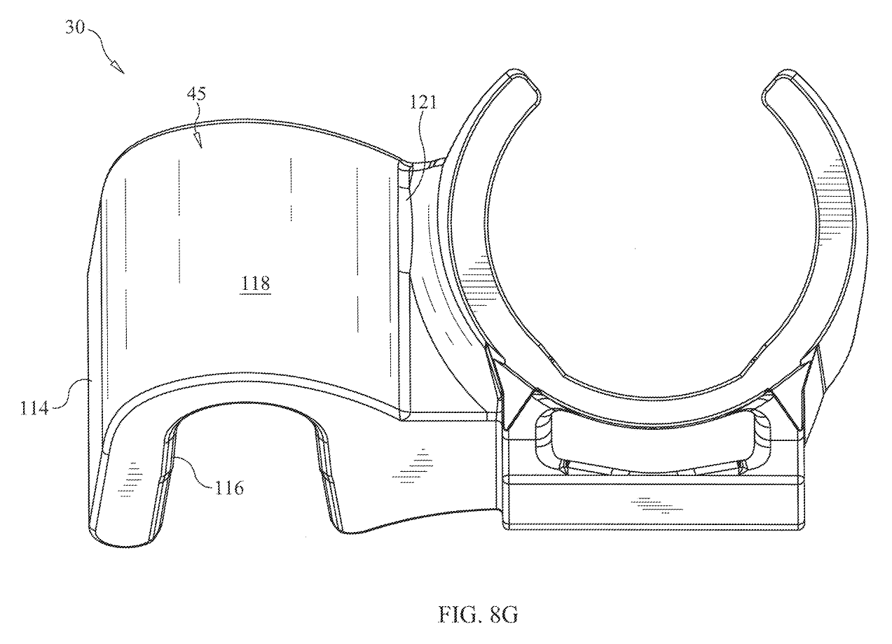

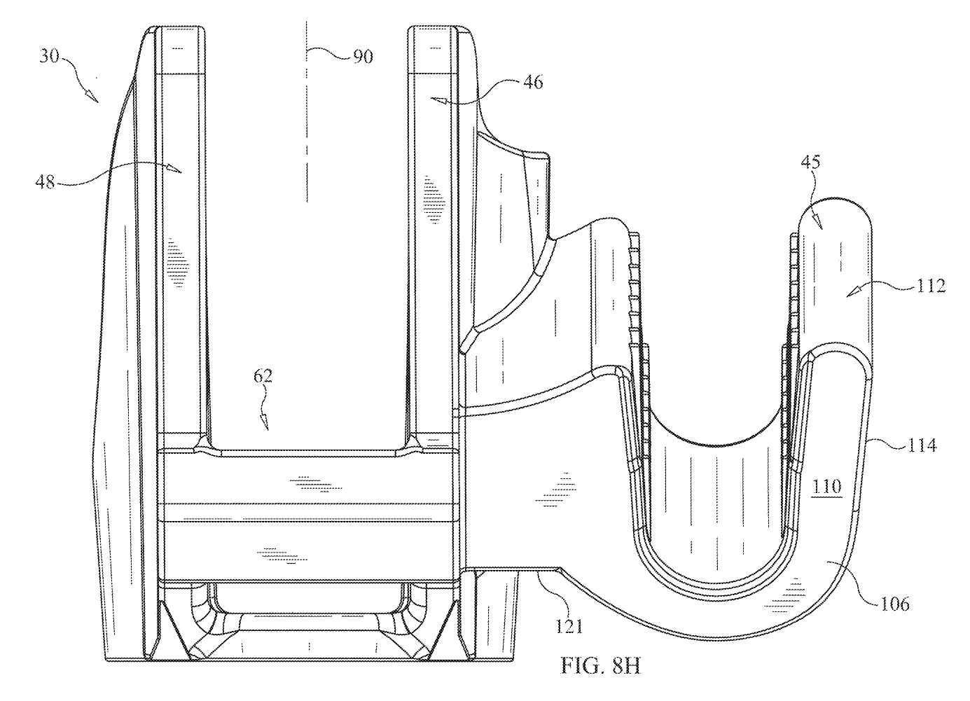

FIG. 8A-FIG. 8E depict another exemplary colonoscope handle attachment device generally at 30. Device 30 is may be a unibody that is integrally extruded, molded, printed, or additively manufactured, removably machined, or formed as a unitary, monolithic member substantially fabricated from a semi-rigid, manmade, material. In one example, polymers, such as hardened plastics, may form a substantial majority of the components or elements used to fabricate the device 30 and the various components integrally formed, molded, or extruded therewith. Device 30 should withstand typical colonoscopy procedure forces performed by a gastroenterologist maneuvering the handle of the device 30 attached to the colonoscope. While it is contemplated device 30 and its additional components described herein are uniformly and integrally extruded, molded, or formed, it is entirely possible that the components of the tool body be formed separately from alternative materials as one having routine skill in the art would understand. In another example, device 30 may be formed from a semi-flexible elastomeric material or rubber material configured to withstand slight deformation upon impact or bending by the operator (i.e., a gastroenterologist). Furthermore, while the components of the device 30 are discussed below individually, it is to be clearly understood that the components and their corresponding reference elements of the tool body are portions, regions, or surfaces of the body and all form a respective element or component of the unitary device 30. Thus, while the components may be discussed individually and identified relative to other elements or components of the tool body, in this exemplary embodiment, there is a single tool body having the below described portions, regions, or surfaces.

With continued reference to FIG. 8A-FIG. 8H, the device 30 includes an upper end 32 opposite a lower end 34 defining a longitudinal direction therebetween. The device 30 includes a forward end 36 opposite a rear end 38 defining a transverse direction therebetween, which is perpendicular to the longitudinal direction. Device 30 further includes a first side 40 and a second side 42 defining a lateral direction therebetween that is perpendicular to the longitudinal direction and perpendicular to the transverse direction.

Device 30 includes a semi-annular body or ring 44 and an attached hook 45. The body or ring 44 includes a first arm 46 and a second arm 48 extending in a longitudinal direction from an upper end to a lower end. More particularly, first arm 46 includes an upper end 50 and a lower end 52. First arm 46 further includes parallel sidewalls defining a thickness of the first arm 46 aligned in the lateral direction. A laterally aligned through aperture 54 extends fully through the width of the first arm 46 adjacent its upper end 50. The second arm 48 is similarly constructed as having an upper end 56 and a lower end 58 aligned in the longitudinal direction and substantially parallel with the respective ends 50, 52 of the first arm 46. Second arm 48 includes planar sidewalls aligned in a longitudinal direction that define a width oriented in the lateral direction of the second arm 48. In one particular embodiment, the width of the second arm 48 is equal to the width of the first arm 46. An aperture 60 is aligned in the lateral direction entirely through the width of the second arm 48. In one particular embodiment, aperture 60 is coaxially centered with aperture 54. As will be described in greater detail below, the pin 409 is configured to be inserted through aperture 54 and aperture 60 in order to releasably secure device 30 to the handle of a colonoscope. Then, while secured to the colonoscope, the hook 45 can be used to secure a portion of the colonoscope or other components in order to free up one hand of the physician. Notably and with respect to first arm 46 and second arm 48, these arms 46, 48 may be referred to as “third” and “fourth” arms respectively in the appended claims as the term “arms” is similarly used to described arms 72, 74 on the body 44 (discussed in greater detail below). Arms 72, 74 may be at least semi-flexible to allow the device 30 to releasably connect with the handle of the colonoscope 450.

A cross member 62 extends laterally between the first arm 46 and the second arm 48 adjacent the respective lower ends 52,58 thereof. Cross member 62 may include an upper portion 64 and a lower portion 66. Upper portion 64 may be angled relative to the lower portion 64. The angle between the upper portion 64 and the lower 66 may be an obtuse angle relative to the lateral axis extending through the cross member 62. Lower portion 66, when viewed from the front (FIG. 8F) further defines a first tapered wall 68 and a second tapered wall 70 that taper downwardly and rearward towards a longitudinal axis. The lower ends of the tapered wall 68 and tapered wall 70 are coplanar along a similar transverse and lateral plane.

Body 44 includes a first semi-arcuate arm 72 and a second semi-arcuate arm 74. Semi-arcuate arm 72 is connected with first arm 46 and the semi-arcuate arm 74 is connected with the second arm 48. First semi-arcuate arm 72 extends forwardly from the first arm 46 in a curved manner towards a terminal forward end 76. The second semi-arcuate arm 74 extends forwardly from the second arm 48 in a curved manner to a forward terminal end 78. An entrance opening 80 is defined between the respective terminal ends 76,78. Semi-arcuate arms collectively define an inner surface and an outer surface of the body 44. More particularly, first semi-arcuate arm 72 includes a concave inner surface 82 and a convex outer surface 84. Semi-arcuate arm 74 includes a concave inner surface 86 opposite a convex outer surface 88.

First arm 72 extends approximately 110 degrees around the circumference of an imaginary circle centered about longitudinal axis 90 between the terminal end 76 and a rear end 92. Similarly, second arm 74 extends approximately 110 degrees around the imaginary circumference of a circle centered about longitudinal axis 90 between the terminal end 78 and a rear end 94. A lower cross member 96 extends arcuately in a curved manner complementary to that of the first arm 72 and second arm 74 between the respective rear ends 92,94. Lower cross member 96 extends approximately 60 degrees around the imaginary circle circumference centered about longitudinal axis 90. Thus, the extent of the body 44 extending around the longitudinal axis 90 is in a range from about 270 degrees to about 300 degrees.

First arm 72 includes a top wall 98 that extends forwardly from an integral connection with respective arms 46,48. Top wall 98 extends forwardly to a rounded corner 100 and then extends downwardly along a vertical surface 102 which concavely curves near the lower end to forwardly bias the terminal end walls 76 forwardly from vertical surface 102.

Second arm 74 includes an elongated S-shaped wall 103 that extends in an elongated S-shaped curved manner from the upper end towards the lower end terminating in a connection with the terminal wall 78.

Lower cross member 96, together with the first arm 46 and the second arm 48, defines an aperture 101 that is generally rectangular in shape and disposed below the cross member 62 and above the lower cross member 96.

Hook 45 includes a first end 104 and a second end 106 defining a length therebetween. First end 104 is defined by a substantially U-shaped surface 108 and the second end 106 is defined by a substantially U-shaped surface 110. First end 104 defining U-shaped surface 108 is disposed rearward from the foremost terminal ends 76, 78 of the arms 72, 74 respectively. Hook 45 includes a U-shaped wall extending between surface 108 and surface 110. The U-shaped wall of hook 45 includes an outer arm 112 having a substantially planar outer surface 114 that is generally orthogonal to U-shaped surface 108 and U-shaped surface 110. Outer arm 112 further includes an inner surface 116 that is generally parallel to outer surface 114. Hook 45 further includes a convexly curved lower surface 118 that extends downwardly in a longitudinal direction from the outer surface 114 and upwardly towards a connection 121 with the arm 72 of the body 44. The connection 121 of hook 45 to body 44 offsets the U-shaped hook 45 off to the first side 40 of the device 30. The connection 121 orients the hook 45 in a cantilevered manner offset to the first side 40 of the body 44.

Hook 45 defines a U-shaped channel 120 extending along a hook axis 122. In one orientation the lower concave portion (concave surface 128) of the U-shaped channel 120 is centered along the hook axis 122. Hook axis 122 is offset from longitudinal axis 90. In one particular embodiment, the hook axis 122 is not parallel to longitudinal axis 90 associated with the body 44. Hook axis 122 may be angled, as represented by angle 124, relative to the longitudinal axis 90. In one particular embodiment, hook axis 122 that is offset from the longitudinal axis 90 is tilted, or canted, or angled in an angle in a range from about 45 degrees to about 75 degrees. More particularly, one particular embodiment may provide that angle 124 is 60 degrees between the hook axis 122 and the longitudinal axis 90. Further, in one particular embodiment, the range of angle 124 may be advantageous to enable some criticality to the operation of the device in order to secure the portion 411 of the colonoscope to the hook 45 at an optimum position to free up the hand of the surgeon while positioning portion 411 at a position that does not interfere with the hand holding the colonoscope.

Outer arm 112 may be semi-flexible in a transverse direction to bend away from and towards hook axis 122. Inasmuch as outer arm may be integrally formed with the other portions of hook 45, the other portions of hook 45 may be semi-flexible as well. The flexibility of the outer arm 112 enable it to bendably flex to couple portions of the colonoscope with the hook 45.

In order to assist securing portions of the colonoscope to hook 45 offset from ring or body 44, the inner surface 116 of the U-shaped hook 45 may include a plurality of angled inwardly extending detents 126. The detents 126 may be embodied as elongated inwardly extending protrusions that are oriented at a non-perpendicular and non-parallel angle relative to hook axis 122. In one particular embodiment, there may be two rows of detents 126 along the inner surface 116 of the U-shaped channel 120 that are oriented relative to the hook axis 122 in a manner that biases a portion 411 of the colonoscope deeper into the channel 120 downwardly towards the upwardly facing concave surface 128 of the hook 45. Detents 126 may be configured a plurality of detents aligned in a row along a length of the inner surface of the hook 45 adapted to bias the sub-component of the colonoscope or the another component towards a lower concave portion of the inner surface of the hook. Additionally, as shown in the figures, there may be at least two rows of detents aligned along the length of the inner surface of the hook 45 adapted to bias the sub-component of the colonoscope or the another component towards the lower concave portion of the inner surface of the hook. In one particular example, the two rows of beneficial may extend inwardly towards hook axis 122 at different lengths. For example, the lower row of detents may extend inwardly towards hook axis 122 farther than the upper row of detents. This different extension of the rows of detents may assist, in accordance with one example, the hook to accommodate different size (i.e., external diameter) scopes that are held by the hook. For example, an adult-sized scope having an external diameter of about 15 mm can be held in place by the first or upper row of detents in the hook and a child-sized scope having an external diameter of about 11 mm can be held in place by the second or lower row of detents in the hook.

In one particular embodiment, the detents 126 are angled downwardly towards the second end 106 of the hook 45 when viewed from a first side elevation view (FIG. 86). Further, the orientation angle of the detents may be longitudinally elongated at the same angle of orientation (angle 124) as the longitudinally axis 90 relative to the hook axis 122. Stated otherwise, if angle 124 is equal to about 60 degrees between the hook axis 122 and the longitudinal axis 90, then the angle at which an axis intersecting the elongated detents 126 intersecting the hook axis 122 would also equal angle 124. In this situation, the detents 126 may be considered to be offset parallel to a longitudinal axis 90.

In accordance with another aspect of the present disclosure, the attachment devices disclosed herein may be formed with various connectors or openings, slots, or recesses that are configured to enable the devices to connect with secondary components to assist with the colonoscopy procedure. For example, the exterior surface of the body 44 or the hook 45 could be formed with a slot, cavity, or recess configuration to receive a portion of an additional component or sub-component therein. Alternatively, the exterior surface of the body or the hook 45 could be formed with a protruding connector that is inserted into to a portion of an additional component that assist the physician during the colonoscopy. In another example, other structural configurations could be part of the device, on either the body 44 or the hook 45 (or both) that complement a component or a portion of a component on an additional device so that the attachment devices presented herein could carry the additional device to assist the physician during a medical procedure.

When device 30 is connected to the colonoscope 450, the first and second arms 72, 74 extend around a portion of the handle of the colonoscope. More particularly, the first and second arms 72, 74 extend partially around an outer circumference of the handle of the colonoscope 450. In one embodiment, the first and second arms 72, 74 are disposed closely adjacent the adjustment ring 413. In this instance the lower surface of the arms 72, 74 could directly contact an end of the 413. Further the lower surface of the arms 72, 74 may directly contact a proximal end of the adjustment ring such that the device 30 is disposed closer to the physician than the adjustment ring 413. Further it is envisioned that the portion of the colonoscope 450 that connects with hook 45 is the shaft 411 of the endoscope.

To attach the device 30 to the colonoscope 450, a physician or another operator will maneuver the arms 72,74 outwardly to expand the opening 80 between the terminal ends 76,78 so device 30 may be snapped onto the colonoscope 450. The ability of arms 72,74 to snap onto the handle of the colonoscope enable the colonoscope handle to occupy the area defined by the inner surfaces 82,86 thereof. The arms 72,74 are resilient to deformation and as such they return to their neutral position to releasably attach or couple body 44 to the handle of the colonoscope 450. Thereafter, the physician may couple or attach another portion of the colonoscope 450 (or a sub-component thereof) to the hook 45. To accomplish this coupling relationship, a user will flexibly bend the outer arm 112 of hook 45 outwardly and away from axis 122. This will widen the space of the U-shaped channel 120 and allow the shaft 411 (or another component) to be moved towards the concave surface 128 defining the lowermost portion of channel 120. When the shaft 411 (or other portion of the colonoscope) is disposed closely adjacent the concave portion 128 of hook 45, the detents 126 secure the shaft 411 in position so that it is effectively locked to the hook 45 until the physician is ready to release the connection of the shaft 411 relative to the hook 45. Stated otherwise, detents 126 preclude movement of the second portion (i.e., shaft 411) of the colonoscope 450 relative to the concave inner surface 128 of the hook 45.

Hook 45 may provide another advantage of enabling the portions of the colonoscope, such as the endoscope, that are supported by the hook to be centered within the colon or intestine during the colonoscopy procedure. The centering of the portions of the colonoscope within the colon may be accomplished by the positioning and angling of the hook axis 122 relative to the longitudinal axis of the body 44. Further, the offset established by connection 121 may further assist to center portions of the colonoscope within the colon or intestine while device 30 is attached to the handle of the colonoscope.

In some embodiments, the attachment devices described herein can be made of a plastic material. In some embodiments, the attachment devices described herein can be single use.

Advantageously, the attachment devices described herein allow for a quick mount and dismount from the colonoscope. For example, mounting can take less than 10 seconds, such as 2-3 seconds.

The attachment devices described herein advantageously help the operator of a colonoscope to intubate difficult segments of the colon. They can also help with negotiations if sharp angulation of tortuous colon and stabilization of the tip of the colon during biopsy, polypectomy, injection, etc. The devices also allow a smooth withdrawal of the colonoscope. Use of the attachment devices described herein can shorten intubation time of the colonic cecum, increase the rate of the intubation of the cecum, increase the quality of colonic inspection, and increase the detection rate of colonic polyps.

It should be understood that any feature described herein with respect to one embodiment can be substituted for or combined with any feature described herein with respect to another embodiment.

Also, various inventive concepts may be embodied as one or more methods, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

While various inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

The articles “a” and “an,” as used herein in the specification and in the claims, unless clearly indicated to the contrary, should be understood to mean “at least one.” The phrase “and/or,” as used herein in the specification and in the claims (if at all), should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc. As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Spatially relative terms, such as “under”, “below”, “lower”, “over”, “upper”, “above”, “behind”, “in front of”, and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal”, “lateral”, “transverse”, “longitudinal”, and the like are used herein for the purpose of explanation only unless specifically indicated otherwise.

Although the terms “first” and “second” may be used herein to describe various features/elements, these features/elements should not be limited by these terms, unless the context indicates otherwise. These terms may be used to distinguish one feature/element from another feature/element. Thus, a first feature/element discussed herein could be termed a second feature/element, and similarly, a second feature/element discussed herein could be termed a first feature/element without departing from the teachings of the present invention.

An embodiment is an implementation or example of the present disclosure. Reference in the specification to “an embodiment,” “one embodiment,” “some embodiments,” “one particular embodiment,” or “other embodiments,” or the like, means that a particular feature, structure, or characteristic described in connection with the embodiments is included in at least some embodiments, but not necessarily all embodiments, of the invention or present disclosure. The various appearances “an embodiment,” “one embodiment,” “some embodiments,” “one particular embodiment,” or “other embodiments,” or the like, are not necessarily all referring to the same embodiments.

If this specification states a component, feature, structure, or characteristic “may”, “might”, or “could” be included, that particular component, feature, structure, or characteristic is not required to be included. If the specification or claim refers to “a” or “an” element, that does not mean there is only one of the element. If the specification or claims refer to “an additional” element, that does not preclude there being more than one of the additional element.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical range recited herein is intended to include all sub-ranges subsumed therein.

Additionally, any method of performing the present disclosure may occur in a sequence different than those described herein. Accordingly, no sequence of the method should be read as a limitation unless explicitly stated. It is recognizable that performing some of the steps of the method in a different order could achieve a similar result.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures.

In the foregoing description, certain terms have been used for brevity, clearness, and understanding. No unnecessary limitations are to be implied therefrom beyond the requirement of the prior art because such terms are used for descriptive purposes and are intended to be broadly construed.

Moreover, the description and illustration of various embodiments of the disclosure are examples and the disclosure is not limited to the exact details shown or described.

What is claimed:

1. An attachment device for connection with a colonoscope comprising:
a body extending along a longitudinal axis sized to connect with a colonoscope; and
a hook coupled to body adapted to carry a sub-component of the colonoscope or another component used during a medical procedure.

2. The attachment device of claim 1, further comprising:
an inner surface of the hook defining a U-shaped channel.

3. The attachment device of claim 2, further comprising:
a lower concave portion of the inner surface of the hook;
a hook axis, wherein the lower concave portion extends around the hook axis.

4. The attachment device of claim 3, wherein the hook axis is oriented relative to the longitudinal axis at an angle in a range from about 45 degrees to about 75 degrees.

5. The attachment device of claim 4, wherein the angle at which the hook axis is oriented relative to the longitudinal axis is about 60 degrees.

6. The attachment device of claim 2, further comprising:
at least one detent extending from the inner surface of the hook.

7. The attachment device of claim 6, further comprising:
a plurality of detents aligned in a row along a length of the inner surface of the hook adapted to retain the sub-component of the colonoscope or the another component near the inner surface of the hook.

8. The attachment device of claim 7, further comprising:
at least two rows of detents aligned along the length of the inner surface of the hook adapted to bias the sub-component of the colonoscope or the another component towards the lower concave portion of the inner surface of the hook.

9. The attachment device of claim 8, wherein at least one detent in one of the at least two rows is elongated relative to the longitudinal axis.

10. The attachment device of claim 8, wherein at least one detent in one of the at least two rows has a length that extends inwardly towards a hook axis that is greater than other lengths of other detents from the plurality of detents.

11. The attachment device of claim 1, further comprising:
a first arm and a second arm defining a semi-annular configuration of the body; and
a third arm and a fourth arm extending longitudinally along an exterior length of the body; and
a transverse aperture formed in each of the third arm and the fourth arm adapted to receive a pin therethrough adapted to couple the attachment device to the colonoscope.

12. The attachment device of claim 1, further comprising a first arm on the body that arm includes:
a top wall that extends forwardly to a corner; and
a surface extending longitudinally from the corner that is concavely curved near a lower end thereof to position a terminal end wall forwardly from the surface.

13. The attachment device of claim 1, further comprising a second arm on the body that arm includes:
an S-shaped wall that is longitudinally elongated from an upper end to a lower end that terminates at a terminal wall of the second arm, and the lower end is disposed forward from the upper end.

14. The attachment device of claim 1, further comprising:
an integral connection of the hook to the body that offsets the hook off to a first side the attachment device in a cantilevered manner.

15. The attachment device of claim 1, further comprising:
a forward end of the hook positioned rearward from a forward end of the body.

16. The attachment device of claim 1, further comprising:
an upper end of the hook positioned below an upper end of the body; and
a lower end of the hook positioned above a lower end of the body.

17. A method comprising:
coupling a body of an attachment device to a first portion of a colonoscope; and
coupling a second portion of the colonoscope to a hook on the attachment device.

18. The method of claim 17, further comprising:
moving the second portion of the colonoscope towards a concave inner surface on the hook; and
precluding movement of the second portion of the colonoscope relative to the concave inner surface of the hook.

19. The method of claim 17, further comprising:
orienting an axis of the second portion of the colonoscope coupled to the hook at an angle in a range from about 45 degrees to 75 degrees relative to a longitudinal axis of the body.

20. The method of claim 17, further comprising:
connecting a secondary component to an exterior surface of one of the body and the hook.
